(19) United States
(12) Patent Application Publication
(10) Pub. No.: US 2005/0211249 A1
(43) Pub. Date: Sep. 29, 2005

(54) Ventilator method, ventilator and memory medium

(76) Inventors: Mirko Wagner, Oberkirch-Nussbach (DE); Siegfried Haussler, Freiburg (DE)

Correspondence Address: KING & SCHICKLI, PLLC, 247 NORTH BROADWAY, LEXINGTON, KY, 40507, US

(21) Appl. No.: 11/132,695

(22) Filed: May 19, 2005

Related U.S. Application Data
(63) Continuation of application No. PCT/DE03/03591, filed on Oct. 29, 2003.

(30) Foreign Application Priority Data
Nov. 19, 2002 (DE) 102 53 946.4

(51) Int. Cl. A62B 7/00; A61M 16/00

(52) U.S. Cl. 128/204.23; 128/204.18

(57) ABSTRACT

The invention relates to a method for a ventilator, wherein a first polynomial is fitted to the time history of a respiratory flow. The invention moreover relates to a method for a ventilator, wherein the derivative of the respiratory flow with respect to time is estimated and a second polynomial is fitted to the derivative. The invention moreover relates to a ventilator, in particular to a CPAP-apparatus, for performing this method, as well as to a memory medium comprising corresponding programs.

### CROSS REFERENCE TO RELATED CO-PENDING APPLICATIONS

This application is a continuation of international application number PCT/DE03/03591 (publication number: WO 2004/045669 A2) filed on Oct. 29, 2003 and entitled VENTILATOR METHOD, VENTILATOR AND MEMORY MEDIUM and claims the benefit of the above-mentioned international application and the corresponding German national patent application number 102 53 946.4-09 filed on Nov. 19, 2002 and entitled VERFAHREN FOR EIN BEATMUNGSGERÄT, BEATMUNGSGERÄT SOWIE SPEICHERMEDIUM the contents of which are expressly incorporated herein by reference.

### FIELD OF THE INVENTION

The invention relates to a method for a ventilator as well as a ventilator and a memory medium. The invention in particular relates to the evaluation of respiratory cycles by extracting therapy-relevant information.

### BACKGROUND OF THE INVENTION

Known are ventilators or respirators for the mechanical artificial respiration for all forms of an oxygen deficiency state. They are, inter alia, applied for the long-time respiration, with a distinction being made between three basic types, depending on the switching mechanism from inspiration to expiration, namely pressure-controlled, volume-controlled and time-controlled ventilators. The ventilator of the latest type are provided with technical, usually electronically controlled devices which allow a respiratory type that satisfies the needs of patients. The inspiration time can, for example, be prolonged up to three times the expiration time, a pressure respiration may be performed, and the ventilator may be “triggered” by the patient, whereby already weak respirations are pulse-generating for the mechanical support (Roche Medical Dictionary, 4^{th} Edition, edited by Hoffmann-La Roche AG and Urban & Fischer, Urban & Fischer, Munich, Stuttgart, Jena, Lübeck, Ulm).

Additionally known are apparatus for performing the CPAP therapy (continuous positive airway pressure) which, in this application, are also designated as ventilator. The CPAP therapy is described in Chest. Volume No. 110, pages 1077-1088, October 1996 and in Sleep, Volume No. 19, pages 184-188. A CPAP-apparatus applies, by means of a compressor, preferably via a humidifier, via a hose and a nose mask, a positive overpressure up to approximately 30 mbar to the respiratory tract of the patient. This positive pressure is to ensure that the upper respiratory tract remains fully opened during the whole night, so that no obstructive apneas will occur (DE 198 49 571 A1).

FIG. 1 shows a CPAP-apparatus 1 and a patient 19. The CPAP-apparatus comprises a compressor 4, a respiratory hose 9, a respiratory mask 18, a pressure sensor 11 and a flow sensor 16. For the generation of an positive pressure the compressor 4 includes a turbine 8. The turbine is also designated as blower, blower unit, compressor, ventilator or fan. In this patent these terms are used synonymously. In the CPAP-apparatus as shown the pressure sensor 11 is accommodated in the compressor housing. In the mask, or in the proximity of the mask, one or several small holes 2 are provided which allow the formation of an air flow from the compressor to the holes 2 on the time average. This prevents the accumulation of CO₂ in the respiratory hose 9 and allows the supply of the patient with oxygen.

The speed of the turbine 8 is controlled by a microcontroller 5 such that the actual pressure measured with the pressure sensor 11 corresponds to a predetermined target pressure. The target pressure is typically preadjusted under the supervision of a medical practitioner and is called titration pressure. The flow sensor can, for example, be a sensor comprising a heating filament 17 which supplies its measuring signal via an instrument lead to the microcontroller in the compressor housing. In another configuration of the CPAP-apparatus a contraction may be provided in the respiratory hose for measuring the respiratory flow. The microcontroller may also take charge of the pressure control.

In the course of the therapy it is necessary to check the compatibility of the apparatus and the adjusted CPAP-pressure. Typically, a patient spends, for this purpose, a control night in a sleeping laboratory.

It has been found that the patients felt the positive pressure generated by the CPAP-apparatus as an unpleasant resistance against which they had to expire. Therefore, control methods for CPAP-apparatus were developed which reduce the target pressure as much as possible. Such a method of controlling the target pressure is described in WO 94/23780. If no respiratory obstructions occur during sleep the pressure is gradually reduced. If sleep disorders such as apneas, hypopneas or snoring occur the pressure is increased.

Similar methods are described in U.S. Pat. No. 5,335,654, EP 0 612 257 B1, WO 99/24099 and EP 0 934 723 A1.

For reducing the unpleasantly felt positive pressure, moreover, BiPAP-apparatus and multilevel apparatus were developed. Such an apparatus is described in DE 691 32 030 T2. The pressure is raised during inspiration by a valve and is reduced during expiration.

According to U.S. Pat. No. 5,740,795 the respiratory flow signal is supplied to a band-limited differentiator. If the output signal of the differentiator exceeds an inspiration threshold or remains under an expiration threshold an expiration detection signal or an inspiration detection signal, respectively, is determined.

Another control method for CPAP-apparatus is described in DE 101 18 968. DE 101 18 968 is included in this application by reference. Initially, the control method calculates features from a measured respiratory flow curve and a measured actual pressure curve of a CPAP-apparatus. Specific combinations of the features are combined to form detectors. Flags are set in the detectors once they detect an event. The control method then alters the target pressure on the basis of the event flag of the detectors.

The features include the expiration time, a backward correlation, a mean inspiration volume, a mean curvature of the respiratory flow during the inspiration as well as the frequency of zero crossings in the alternating component of the actual CPAP pressure.

When changing from inspiration to expiration a distinct edge can be recognized in the time history of the respiratory flow which is used for the detection of individual respirations. The local maximums of the first derivative of the respiratory flow with respect to time correspond to the maximum gradient of the respiratory flow when changing between inspiration and expiration. The beginning of the inspiration is looked for from the end of the inspiration by looking for the first local minimum in the estimated derivative. The expiration time results as time difference between a minimum of the estimated derivative and the preceding maximum of the estimated derivative. Due to noise in the respiratory flow curve the respiratory flow curve is not merely differentiated, but is additionally lowpass-filtered. The derivative and lowpass-filtering are accomplished in a filtering step by appropriately selecting the coefficients of a digital filter. “Estimating the derivative” will in this application be used as a generic term for derivative and derivative with lowpass-filtering.

For calculating the mean curvature of the respiratory flow during the inspiration the estimated first derivative of the respiratory flow with respect to time during the inspiration is used. Subsequently a straight line is fitted to the estimated first derivative. The gradient of this fitted straight line results in the mean curvature of the inspiration.

According to the teaching of DE 101 18 968 an apnea detector, a hypopnea detector and a respiratory flow limitation detector are calculated from the features as an indication of a pressure increase, and a normal detector as an indication of a stable respiration and a possible pressure reduction.

For recognizing a stable respiration the normal detector uses the backward correlation. The respiration is stable if the target pressure was not altered during a predetermined time, e.g. 180 sec., and the backward correlation during this time is, for example, ≧0.86.

### SUMMARY OF THE INVENTION

According to an embodiment of the invention a ventilator method is provided. A respiratory air flow is repeatedly measured during the operation of the ventilator at several points in time. The measured samples of the respiratory flow are stored. A first polynomial is fitted to the time history of the respiratory flow.

According to another embodiment of the invention another ventilator method is provided. This method also comprises repeatedly measuring the respiratory flow during the operation of the ventilator at several points in time. Further, the measured points of the respiratory flow are stored. The derivative of the respiratory flow with respect to time is estimated. A second polynomial of the third order is fitted to the derivative.

According to a further embodiment of the invention a ventilator is provided. The ventilator comprises a flow sensor, a command memory and a central processing unit. The flow sensor repeatedly measures a respiratory air flow during the operation of the ventilator at several points in time. The central processing unit processes commands stored in the command memory, so that the measured samples of the respiratory flow are stored, and the central processing unit fits a first polynomial to the time history of the respiratory flow.

According to yet a further embodiment of the invention a ventilator is provided. The ventilator comprises a flow sensor, a command memory and a central processing unit. The flow sensor repeatedly measures a respiratory air flow during the operation of the ventilator at several points in time. The central processing unit processes commands stored in the command memory, so that the measured samples of the respiratory flow are stored, the derivative of the respiratory flow with respect to time is estimated, and a second polynomial of the third order is fitted to the derivative.

According to another embodiment of the invention a memory medium for use with a ventilator is provided. The memory medium comprises data constituting coefficients of polynomials fitted to data obtained from respiratory flow data provided by the ventilator.

According to a further embodiment of the invention a memory medium for use with a ventilator is provided. The memory medium comprises data constituting commands to be processed by a central processing unit of the ventilator. The ventilator comprises a flow sensor for repeatedly measuring a respiratory air flow during the operation of the ventilator at several points in time. The commands instruct the central processing unit to store the measured samples of the respiratory flow and to fit a first polynomial to the time history of the respiratory flow.

An advantage of the fitting of a polynomial to the time history of a respiratory flow curve or the derivative thereof resides in a considerable reduction of data, a lowpass-filtering for the cancellation of noise as well as the extraction of therapy-relevant information, i.e. information characterizing the state of the patient. In an advantageous manner this information can be used by the ventilator at once, e.g. for correcting a target pressure.

Moreover, the polynomial can be stored in form of its coefficients and can be evaluated by a medical practitioner off-line for controlling the therapy. With a small extra expenditure of electronic components this form of evaluation renders control nights in sleeping laboratories superfluous, which entails a cost advantage for the health insurance funds. The data obtained during a control night can thus be recorded in the home environment of the patient which is more convenient to him.

A polynomial of the fourth order fitted to the respiratory flow history during an inspiration phase still includes the therapy-relevant information and thus constitutes a good compromise between the reduction of data and the maintenance of therapy-relevant information.

The fitting of the polynomial to an inspiration phase of the respiratory flow history is in particular advantageous for the CPAP therapy because the pressure in the respiratory tract is lower than the ambient pressure during the inspiration phase only, so that the respiratory tract collapses and may result in an apnea.

The provision of an interface for an external memory unit makes the ventilator easier to handle because the patient need not take the entire apparatus, but only the external memory unit to the doctor for controlling the therapy.

Even more convenient is the remote data transmission by modem, as no physical object has to be moved.

The three, possibly complex, zeros of the derivative of a polynomial of the fourth order, which was fitted to the time history of the respiratory flow, may advantageously be used to adjust the target pressure. The three, possibly complex, zeros of a polynomial of the third order, which was fitted to the derivative of the respiratory flow history with respect to time, are analogously suited.

One feature for the respiration quality is the imaginary part of the conjugate complex zeros, if available.

An even more stable feature seems to be the area of the triangle formed by the three zeros in the complex plane.

### BRIEF DESCRIPTION OF THE DRAWINGS

Preferred embodiments of the invention will hereinafter be explained in more detail with reference to the enclosed drawings, wherein like numerals represent like parts.

FIG. 1 shows a CPAP-apparatus,

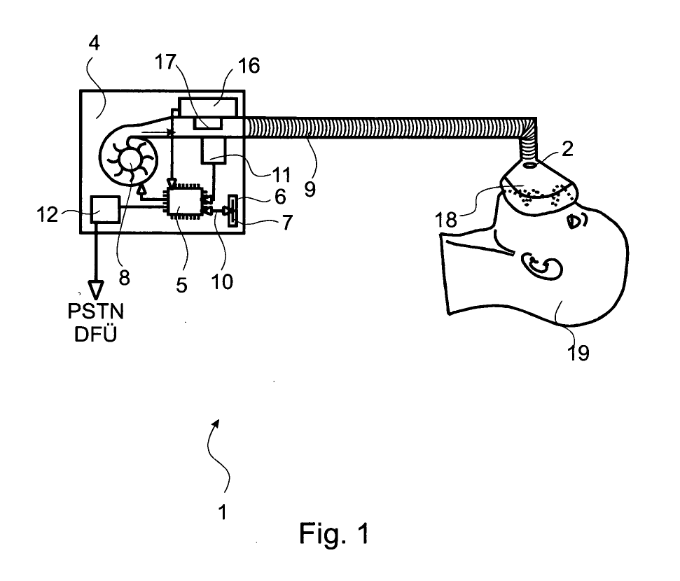

FIG. 2 shows inspiration phases of a respiratory flow curve with regular respiration,

FIG. 3 shows inspiration phases of a respiratory flow curve with flow-limited respiration, and

FIGS. 4 and 5 show flow charts of two methods according to the invention.

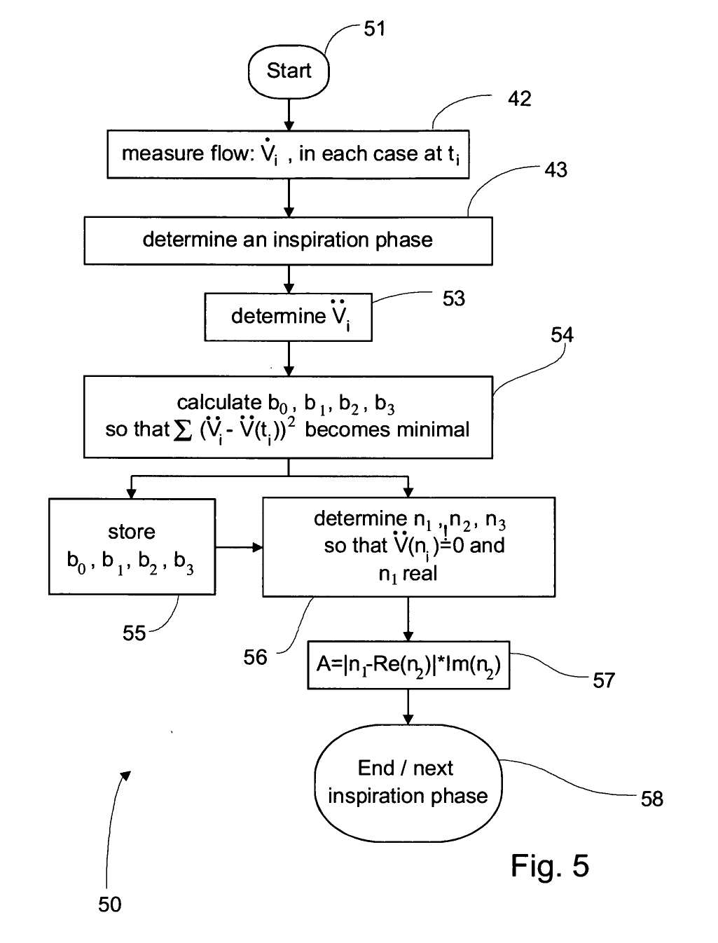

### DETAILED DESCRIPTION OF THE INVENTION

As was mentioned above, a patient usually now and then spends a control night in a sleeping laboratory for the control of the therapy. The information obtained in a control night can substantially be extracted from the recorded flow data. For the control of the therapy it must in particular be decided on the basis of the recorded flow data whether or not the respiratory flow during sleep is limited. Therefore, control nights can be saved with a memory device for flow data on the CPAP-apparatus.

The required volume of data can be estimated as follows: During an average sleeping period of 8 hours corresponding to 28800 s a human being respires approximately 28800 s/3 s=9600 times. With a sampling rate of 100 Hz the number of data thus amounts to 2.88·10⁶ data points per night. This volume of data is too large for being stored in a conventional CPAP-apparatus.

The therapy-relevant information in the different inspiration patterns with normal and flow-limited respiration, respectively, may be filtered out with a real polynomial of the fourth order (equation 1). A polynomial of the fourth order includes sufficient information to describe the different inspiration patterns between normal and flow-limited respiration exactly enough so as to be suited for the therapy control.

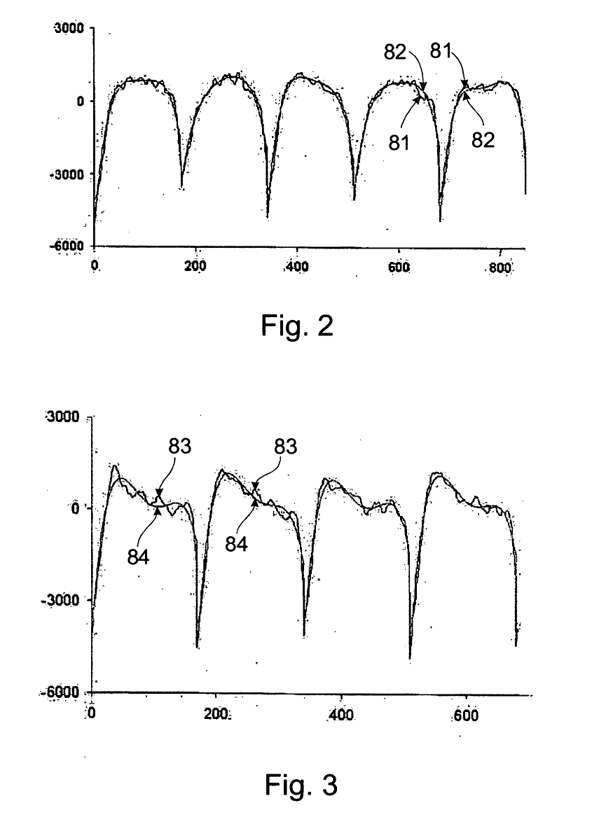

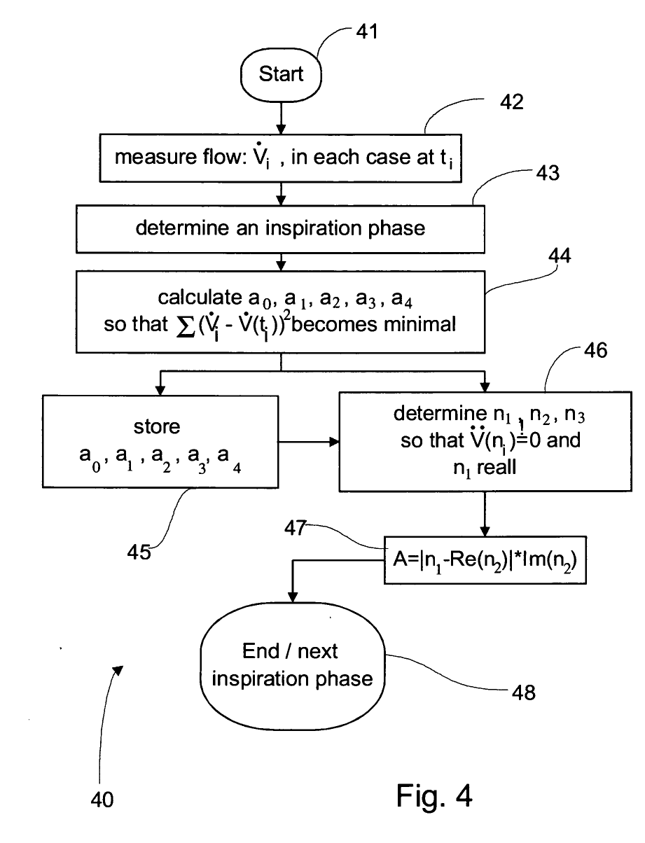

Several inspiration phases of normal and flow-limited respirations are illustrated in FIG. 2 and FIG. 3. The less smooth curves 81 and 83 constitute the measured flow data. The smoother curves 82 and 84 are the polynomials of the fourth order fitted to the flow data. One clearly recognizes that the inspiration phases of normal regular respirations in FIG. 2 are similar to downwardly open parabolas. Contrary to this, the inspiration phases shown in FIG. 3 rather have an angular parallelogram-shaped course. After a maximum after the first fifth of the inspiration phase the respiratory flow slowly drops nearly till the end of the inspiration phase. The drop of the respiratory flow often accelerates only in the last tenth of the inspiration phase. As can be seen from the smoother polynomial curves 84 the respiratory flow extends over a wide portion of the second half of the inspiration phases horizontally and even has a local maximum in the second half of the inspiration phases. In particular in the first, third and fourth of the inspiration phases shown in FIG. 3 have the fitted polynomials two maximums and one local minimum located therebetween.
\dot{V}(t)=a₀+a₁t+a₂t²+a₃t³+a₄t⁴ (1)

In equation 1 \dot{V}(t) is the air flow, t the time, and a₀ to a₄ are selectable polynomial coefficients. For presenting an inspiration phase it is sufficient to store the 5 polynomial coefficients a₀ to a₄. Thus, the memory space requirement per night with 9600 respirations is reduced to 48000 polynomial coefficients instead of 2.88·10⁶ data points. The memory space requirement is thus reduced by a factor 60.

The fitting of the polynomial coefficients a₀ to a₄ may be performed in the usual manner by minimizing the sum of squares of the deviations between measured and calculated flow values in accordance with equation 2. There are known algorithms for this, which accomplish a numerically complex minimum search by solving a linear system of equations. In other embodiments other criteria for the fitting of a polynomial, in particular one of the fourth order, to the respiratory flow history may be used. According to equation 3 in particular the sum of the absolute value of the differences between measured values and polynomial values can be minimized.

$$\begin{matrix}{Σ ( {\dot{V}}_{i} - \dot{V} ( {t}_{i} ) )}^{2} {=}^{!} min & ( 2 ) \\ Σ \vert {\dot{V}}_{i} - \dot{V} ( {t}_{i} ) \vert {=}^{!} min & ( 3 )\end{matrix}$$

A polynomial of the fourth order may have either 1 or 3 extremes. As is known to skilled persons, extremes are found by looking for zeros in the derivative. Equation 4 includes the derivative with respect to time of the polynomial of the fourth order from equation 1. It may be represented as polynomial of the third order with the coefficients b₀ to b₃. The coefficients b₀ to b₃ may be correlated by a coefficient comparison with the coefficients a₁ to a₄.

$$\begin{matrix}\begin{matrix}\frac{ⅆ}{ⅆ t} \dot{V} ( t ) = \ddot{V} ( t ) \\ = {a}_{1} + 2 {a}_{2} t + 3 {a}_{3} {t}^{2} + 4 {a}_{4} {t}^{3} \\ = {b}_{0} + {b}_{1} t + {b}_{2} {t}^{2} + {b}_{3} {t}^{3}\end{matrix} & ( 4 ) \\ \ddot{V} ( t ) = {b}_{0} + {b}_{1} t + {b}_{2} {t}^{2} + {b}_{3} {t}^{3} {=}^{!} 0 & ( 5 )\end{matrix}$$

A polynomial of the third order with real coefficients has three zeros n₁, n₂ and n₃ whereof two may be conjugate complex zeros. Without limiting the generality zero n₁ is to be always real. If three real zeros are found in equation 5 the corresponding polynomial of the fourth order in equation 1 has two local maximums and one local minimum (cf. inspiration pattern in FIG. 3). If merely one real from two conjugate complex zeros is found in equation 5 the corresponding polynomial of the fourth order in equation 1 merely has one maximum. In the latter case the inspiration phases equal downwardly open parabolas (cf. inspiration pattern in FIG. 2).

It has been found that the evaluation of the absolute value of the imaginary parts, the absolute values of which are equally large, of both conjugate complex zeros provides a feature in the sense of DE 101 18 968. The larger the quantity of the imaginary parts, the more stable is the respiration. From this another normal detector according to DE 101 18 968 can be obtained by comparing the quantity of the imaginary parts with a threshold and by determining a normal event, if the quantity of the imaginary parts is above the threshold. The advantage of such a normal detector as compared to the normal detector according to DE 101 18 968, which is based on the backward correlation, resides in that it supplies one event per respiration, i.e. it does not require a plurality of respirations.

According to the present state of knowledge the area of the triangle formed by the three zeros in the complex plane even is a more stable feature in the sense of DE 101 18 968. Should three real zeros result with instable respiration, this area is zero. By calculating the area no case differentiation between three real or only one real zero has to be made.

For the definition of another normal detector this triangular area may be compared with a threshold, with a normal event being present if the threshold is exceeded. Also a so defined normal detector supplies one event per respiration.

The advantage of such features is that they are capable of evaluating the quality of normal respiration. A pressure control mechanism for optimally adjusting the target pressure in a CPAP-apparatus then no longer has to provoke respiratory events, like in DE 102 53 935 B3 and WO 2004/045693 A2, to detect that the target pressure cannot be reduced further. With the above-described features it can rather be recognized still in the normal respiration range underneath which pressure there is a danger of a respiratory flow limitation. The patient is then disturbed by provoked respiratory events during his sleep to a smaller extent.

In another embodiment a polynomial of the third order (see equation 4) may be fitted to the derivative with respect to time or to the estimated derivative with respect to time of the respiratory flow. The derivative of the respiratory flow with respect to time is estimated for the determination of the changes between inspiration and expiration anyhow, so that data on the derivative of the respiratory flow are present. The fitting of a polynomial of the third order is less complex with respect to calculation than the fitting of a polynomial of the fourth order. The fitting of the polynomial to the derivative may be accomplished in accordance with the criteria mentioned in equations 2 and 3 or other criteria. As the fitting of polynomials to measured curves is not a linear operation, slightly different coefficients for the polynomials of the third order will result from exchanging the order of polynomial fitting and derivative, even if identical measurement data are assumed. Thus, also the zeros will be slightly different. This may, however, be compensated by shifting the thresholds in the corresponding normal detectors.

The polynomials of the third order include fewer therapy-relevant information. These polynomials in particular include no information on the mean inspiration volume. If this is not relevant, however, also the coefficients of the polynomials of the third order may be recorded and evaluated by a doctor later for controlling the therapy.

A ventilator may comprise a digital signal processor (DSP) and/or a microcontroller 5 for performing the data reduction method. For storing the data an external memory device 7, e.g. in the form of a PCMCIA card, a Smartcard, a memory dongle or a memory stick, may be provided for which the CPAP apparatus may have a plug-in slot 6.

In another embodiment the ventilator may be provided with a modem 12 (modulator demodulator) via which the CPAP-apparatus may transmit the data, e.g. via a public telephone network (PSTN, public switched telephone network), to a computer of a doctor. Moreover, also emergency calls may be made via the modem 12 of the ventilator. It is particularly advantageous to buffer the flow data in the ventilator first so that no active connection via the public telephone network has to be maintained. A reduction of data reduces the line holding time and thus the telephone costs.

In the foregoing, the invention was explained in more detail by means of preferred embodiments. It is, however, obvious for a person skilled in the art that various alterations and modifications may be made without departing from the spirit of the invention. Therefore, the scope of protection is defined by the following claims and their equivalents.

### LIST OF REFERENCE NUMERALS

1 CPAP-apparatus
2 hole
4 compressor
5 microcontroller
6 plug-in slot
7 memory medium
8 turbine
9 respiratory hose
10 data line
11 pressure sensor
12 modem
16 flow sensor
17 heating filament
18 respiratory mask
19 sleeping person
40 flow chart
41-48 steps
50 flow chart
51-58 steps
81 measured respiratory flow curve
82 fitted respiratory flow curve
83 measured respiratory flow curve
84 fitted respiratory flow curve

1. A ventilator method comprising:
repeatedly measuring a respiratory air flow during the operation of the ventilator at several points in time;
storing the measured samples of the respiratory flow; and
fitting a first polynomial to the time history of the respiratory flow.

2. The method according to claim 1, wherein the first polynomial is a polynomial of the fourth order.

3. The method according to claim 1, wherein the measured points of the respiratory flow are subdivided in individual inspiration and expiration phases and the first polynomial is fitted to the points belonging to an inspiration phase.

4. The method according to claim 1, wherein the ventilator transmits the polynomial coefficients of the first polynomial via an interface to an external memory unit.

5. The method according to claim 1, wherein the ventilator transmits the polynomial coefficients of the first polynomial via a modem and a public telephone network (PSTN) to another computer.

6. The method according to claim 2 further comprising:
calculating the derivative of the first polynomial, thereby forming a second polynomial of the third order.

7. A ventilator method comprising:
repeatedly measuring the respiratory flow during the operation of the ventilator at several points in time;
storing the measured points of the respiratory flow;
estimating the derivative of the respiratory flow with respect to time; and
fitting a second polynomial of the third order to the derivative.

8. The method according to claim 7, further comprising:
determining the three zeros of the second polynomial;
evaluating the imaginary part of the two conjugate complex zeros if the second polynomial only has one real zero.

9. The method according to claim 8, wherein further the area of the triangle formed by the three zeros in the complex plane is calculated.

10. A ventilator comprising:
a flow sensor for repeatedly measuring a respiratory air flow during the operation of the ventilator at several points in time;
a command memory; and
a central processing unit for processing commands stored in the command memory, so that the measured samples of the respiratory flow are stored; and
the central processing unit fits a first polynomial to the time history of the respiratory flow.

11. The ventilator of claim 10, wherein the ventilator is a CPAP-apparatus.

12. The ventilator according to claim 10, wherein the first polynomial is a polynomial of the fourth order.

13. The ventilator according to claim 10, wherein the command memory comprises commands instructing the central processing unit to subdivide the measured samples of the respiratory flow in individual inspiration and expiration phases and the first polynomial is fitted to the points belonging to an inspiration phase.

14. The ventilator according to claim 10, wherein the ventilator further comprises an interface for transmitting the polynomial coefficients of the first polynomial to an external memory unit.

15. The method according to claim 10, wherein the ventilator transmits the polynomial coefficients of the first polynomial via a modem and a public telephone network (PSTN) to another computer.

16. A ventilator, comprising:
a flow sensor for repeatedly measuring a respiratory air flow during the operation of the ventilator at several points in time;
a command memory; and
a central processing unit for processing commands stored in the command memory, so that the measured samples of the respiratory flow are stored, the derivative of the respiratory flow with respect to time is estimated, and a second polynomial of the third order is fitted to the derivative.

17. The ventilator according to claim 16, the command memory further comprising commands instructing the central processing unit to determine the three zeros of the second polynomial and evaluate the imaginary part of the two conjugate complex zeros if the second polynomial only has one real zero.

18. The method according to claim 17, the command memory further comprising commands instructing the central processing unit to calculate the area of the triangle formed by the three zeros in the complex plane.

19. A memory medium for use with a ventilator, comprising data constituting coefficients of polynomials fitted to data obtained from respiratory flow data provided by the ventilator.

20. A memory medium for use with a ventilator, the memory medium comprising data constituting commands to be processed by a central processing unit of the ventilator, the ventilator comprising a flow sensor for repeatedly measuring a respiratory air flow during the operation of the ventilator at several points in time; the commands instructing the central processing unit to store the measured samples of the respiratory flow and to fit a first polynomial to the time history of the respiratory flow.
